(19) United States
(12) Patent Application Publication
(10) Pub. No.: US 2020/0030557 A1
(43) Pub. Date: Jan. 30, 2020

(54) MEDICAL TUBES FOR SELECTIVE MECHANICAL VENTILATION OF THE LUNGS

(71) Applicant: Guillermo L. Pol, Coral Gables, FL (US)

(72) Inventor: Guillermo L. Pol, Coral Gables, FL (US)

(21) Appl. No.: 16/525,385

(22) Filed: Jul. 29, 2019

Related U.S. Application Data
(60) Provisional application No. 62/703,969, filed on Jul. 27, 2018.

(51) Int. Cl. A61M 16/04; A61M 16/10; A61B 1/267

(52) U.S. Cl. CPC A61M 16/0404; A61M 16/0434; A61M 16/0486; A61M 16/0475; A61M 2210/1039; A61B 1/2676; A61M 2210/1032; A61M 2210/1035; A61M 16/104

(57) ABSTRACT

An endobronchial tube is provided that includes a medical tube comprising an endotracheal portion and a bronchial portion having a common single lumen extending between a proximal end and a distal end of the tube, a first endotracheal inflatable cuff for sealing against a trachea of a patient, a second endotracheal inflatable cuff positioned distal to the first endotracheal inflatable cuff for sealing against the trachea of the patient; a bronchial inflatable cuff positioned near a distal end of the medical tube after the first endotracheal inflatable cuff and the second endotracheal inflatable cuff, for sealing the left main stem bronchi of the patient, an intraluminal balloon blocker positioned along an inner surface of the bronchial portion for sealing the common single lumen at the distal end of the bronchial portion, and an aperture positioned between the endotracheal portion and the bronchial portion.

### CROSS-REFERENCE TO RELATED APPLICATION(S)

This application claims priority to, and the benefit of, co-pending U.S. Provisional Application No. 62/703,969, filed Jul. 27, 2018, for all subject matter common to both applications. The disclosure of said provisional application is hereby incorporated by reference in its entirety.

### FIELD

The embodiments disclosed herein relate to medical tubes for selective mechanical ventilation of the lungs, and more particularly to single lumen endobronchial tubes for selective mechanical ventilation of the left lung or the right lung.

### BACKGROUND

The body requires a certain volume of air to be inhaled and exhaled to maintain the correct levels of oxygen and carbon dioxide within the tissues. Tissue damage, which leads eventually to death, occurs if the level of oxygen becomes too low or the amount of carbon dioxide becomes too high. The body is therefore critically dependent on breathing to maintain life. In medicine, mechanical ventilation is a method to mechanically assist or replace spontaneous breathing. A medical ventilator moves breathable air into and out of the lungs, to provide the mechanism of breathing for a patient who is physically unable to breathe, or breathing insufficiently. Ventilators are chiefly used in intensive care medicine and emergency medicine (as standalone units) and in anesthesia (as a component of an anesthesia machine).

### SUMMARY

Single lumen endobronchial tubes for selective mechanical ventilation of the left lung or the right lung from the left lung are disclosed herein.

In accordance with some aspects of the present disclosure, an endobronchial tube is provided that includes a medical tube comprising an endotracheal portion and a bronchial portion having a common single lumen extending between a proximal end and a distal end of the tube, wherein the proximal end of the endotracheal portion includes a first opening adapted for connection to an external mechanical ventilation device and the distal end of the bronchial portion includes a second opening adapted for delivery of a medical gas. The tube also includes a first endotracheal inflatable cuff positioned around an external surface of the endotracheal portion and adapted to expand radially outward for sealing against a trachea of a patient and a second endotracheal inflatable cuff positioned around an external surface of the endotracheal portion and located at a position distal to the first endotracheal inflatable cuff, the second endotracheal inflatable cuff adapted to expand radially outward for sealing against the trachea of the patient. The first endotracheal inflatable cuff and the second endotracheal inflatable cuff are tapered. The tube further includes a bronchial inflatable cuff positioned around an external surface of the bronchial portion located near a distal end of the medical tube after the first endotracheal inflatable cuff and the second endotracheal inflatable cuff, the bronchial inflatable cuff adapted to expand radially outward against the left main stem bronchi of the patient, an intraluminal balloon blocker positioned along an inner surface of the bronchial portion and adapted to expand for sealing the common single lumen at the distal end of the bronchial portion, and an aperture positioned between the first tracheal inflatable cuff and the second tracheal inflatable cuff and adapted to deliver an amount of medical gas to the second lung of the patient. The single lumen defines a single passageway that fluidly connects the aperture, the first opening, and the second opening with one another to selectively ventilate the left lung, the right lung, or both lungs of the patient.

In accordance with some aspects of the present disclosure, the first endotracheal inflatable cuff, the second endotracheal inflatable cuff, the bronchial inflatable cuff and the intraluminal balloon blocker are each remotely and selectively inflatable. The first endotracheal inflatable cuff and the second endotracheal inflatable cuff can be tapered in opposite directions toward the aperture. The first endotracheal inflatable cuff and the bronchial inflatable cuff can be inflatable and deflatable via a single inflation channel. The intraluminal balloon blocker can be immediately distal to the aperture. The aperture can be beveled.

In accordance with some aspects of the present disclosure, upon placement of the tube in a trachea of the patient and the left main stem bronchi, selective ventilation of the left lung or the right lung is achievable without a need to move or reposition the tube, wherein to selectively ventilate the left lung of the patient, the first tracheal inflatable cuff, the second tracheal inflatable cuff, and the bronchial inflatable cuff are placed in an inflated position and the intraluminal balloon blocker is placed in a deflated position, and to selectively ventilate the right lung of the patient, the first tracheal inflatable cuff, the bronchial inflatable cuff and the intraluminal balloon blocker are placed in the inflated position and the second tracheal inflatable cuff is placed in the deflated position

In accordance with some aspects of the present disclosure, an endobronchial tube is provided that includes a medical tube comprising an endotracheal portion and a bronchial portion having a common single lumen extending between a proximal end and a distal end of the tube, wherein the proximal end of the endotracheal portion includes a first opening adapted for connection to an external mechanical ventilation device and the distal end of the bronchial portion includes a second opening adapted for delivery of a medical gas and a first endotracheal inflatable cuff positioned around an external surface of the endotracheal portion and adapted to expand radially outward for sealing against a trachea of a patient. The tube also includes a second endotracheal inflatable cuff positioned around an external surface of the endotracheal portion and located at a position distal to the first endotracheal inflatable cuff, the second endotracheal inflatable cuff adapted to expand radially outward for sealing against the trachea of the patient and a bronchial inflatable cuff positioned around an external surface of the bronchial portion located near a distal end of the medical tube after the first endotracheal inflatable cuff and the second endotracheal inflatable cuff, the bronchial inflatable cuff adapted to expand radially outward against the left main stem bronchi of the patient. The tube further includes a beveled aperture positioned between the first tracheal inflatable cuff and the second tracheal inflatable cuff and adapted to deliver an amount of medical gas to the second lung of the patient. The single lumen defines a single passageway that fluidly connects the beveled aperture, the first opening, and the second opening with one another to selectively ventilate the left lung, the right lung, or both lungs of the patient.

In accordance with some aspects of the present disclosure, the tube can further include a distal intraluminal balloon blocker adapted to expand radially outward sealing the common single lumen. The distal intraluminal balloon blocker can be a low volume high pressure member. The tracheal inflatable cuff, the bronchial inflatable cuff and the distal intraluminal balloon blocker can each be remotely and selectively inflatable. The tube can further include a second tracheal inflatable cuff positioned around an external surface of the tracheal portion and adapted to expand radially outward at a respective distal location relative to the aperture. The tube can further include a built-in video camera embedded within the common tube wall.

In accordance with some aspects of the present disclosure, upon placement of the tube in a trachea of the patient and the left main stem bronchi, selective ventilation of the left lung or the right lung is achievable without a need to move or reposition the tube, wherein to selectively ventilate the left lung of the patient, the first tracheal inflatable cuff, the second tracheal inflatable cuff, and the bronchial inflatable cuff are placed in an inflated position and the intraluminal balloon blocker is placed in a deflated position, and to selectively ventilate the right lung of the patient, the first tracheal inflatable cuff, the bronchial inflatable cuff and the intraluminal balloon blocker are placed in the inflated position and the second tracheal inflatable cuff is placed in the deflated position

In accordance with some aspects of the present disclosure, a method for one-lung ventilation of a lung is provided. The method includes inserting a single lumen endobronchial tube into in a trachea of a patient and a left main stem bronchi of a left lung of the patient. The single lumen endobronchial tube includes a medical tube comprising an endotracheal portion and a bronchial portion having a common single lumen extending between a proximal end and a distal end of the tube, wherein the proximal end of the endotracheal portion includes a first opening adapted for connection to an external mechanical ventilation device and the distal end of the bronchial portion includes a second opening adapted for delivery of a medical gas and a first endotracheal inflatable cuff positioned around an external surface of the endotracheal portion and adapted to expand radially outward for sealing against a trachea of a patient. The tube also includes a second endotracheal inflatable cuff positioned around an external surface of the endotracheal portion and located at a position distal to the first endotracheal inflatable cuff, the second endotracheal inflatable cuff adapted to expand radially outward for sealing against the trachea of the patient, wherein the first endotracheal inflatable cuff and the second endotracheal inflatable cuff are tapered and a bronchial inflatable cuff positioned around an external surface of the bronchial portion located near a distal end of the medical tube after the first endotracheal inflatable cuff and the second endotracheal inflatable cuff, the bronchial inflatable cuff adapted to expand radially outward against the left main stem bronchi of the patient. The tube further includes an intraluminal balloon blocker positioned along an inner surface of the bronchial portion and adapted to expand for sealing the common single lumen at the distal end of the bronchial portion and an aperture positioned between the endotracheal portion and the bronchial portion and adapted to deliver an amount of medical gas to the second lung of the patient. The single lumen defines a single passageway that fluidly connects the beveled aperture, the first opening, and the second opening with one another to selectively ventilate the left lung, the right lung, or both lungs of the patient. The method also includes positioning the tube in the pulmonary airway of a patient such that the di bronchial inflatable cuff is in the left main stem bronchus, and the first endotracheal cuff and the second endotracheal inflatable cuff are in the trachea and connecting the proximal end of the medical tube to the external mechanical ventilation device. The method further includes inflating the bronchial inflatable cuff radially outwardly to seal against the surrounding bronchus of the left lung and inflating at least one of the first endotracheal inflatable cuff and the second endotracheal inflatable cuff radially outwardly to seal against the surrounding trachea of the patient.

In accordance with some aspects of the present disclosure, the first endotracheal inflatable cuff and the second endotracheal inflatable cuff are tapered in opposite directions toward the aperture. The aperture can be beveled. The tube can further include a built-in video camera embedded within the tube wall for real-time visualization of the method.

In accordance with some aspects of the present disclosure, upon insertion of the tube into the left main stem bronchi, selective ventilation of the left lung or the right lung is achievable without a need to move or reposition the tube, wherein to selectively ventilate the left lung of the patient, the first tracheal inflatable cuff, the second tracheal inflatable cuff, and the bronchial inflatable cuff are placed in an inflated position and the intraluminal balloon blocker is placed in a deflated position, and to selectively ventilate the right lung of the patient, the first tracheal inflatable cuff, the bronchial inflatable cuff and the intraluminal balloon blocker are placed in the inflated position and the second tracheal inflatable cuff is placed in the deflated position.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1A is a side view of an embodiment of a single lumen endobronchial tube of the present disclosure.

FIG. 1B shows a side view of an embodiment of a single lumen endobronchial tube with tapered cuffs.

FIG. 2A and FIG. 2B are cross-sectional plan views of FIG. 1. FIG. 2A shows a distal intraluminal balloon of the single lumen endobronchial tube in an inflated state. FIG. 2B shows a distal intraluminal balloon of the single lumen endobronchial tube in a deflated state.

FIG. 2C shows a cross-sectional plan view of FIG. 1.

FIG. 2D shows a side view of an embodiment of a single lumen endobronchial tube with a single airflow channel for inflating and deflecting a bronchial cuff and an tracheal cuff together.

FIG. 3A shows a side view of an embodiment of a single lumen endobronchial tube with a beveled aperture and intraluminal balloon placement in relation to the aperture.

FIG. 3B shows a front view of an embodiment of a single lumen endobronchial tube with a beveled aperture and intraluminal balloon placement in relation to the aperture.

FIG. 4 shows a schematic view of the single lumen endobronchial tube of FIG. 1A positioned in a person for the selective ventilation of the left lung.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Mechanical ventilation has become the most commonly used mode of life support in medicine today. Widely used in management of acutely ill surgical and ICU patients, mechanical ventilation can also be used in the chronic support of patients with a wide spectrum of chronic diseases that can cause respiratory failure.

As used herein, the term “anesthesia machine” refers to a machine used by an anesthesiologist to support the administration of anesthesia. The most common type of anesthesia machine, the continuous-flow anesthesia machine, is designed to provide an accurate and continuous supply of medical gases (such as oxygen and nitrous oxide), mixed with an accurate concentration of anesthetic vapor (such as isoflurane), and deliver this to the patient at a safe pressure and flow. Modern machines incorporate a medical ventilator, suction unit, and patient-monitoring devices.

As used herein, the term “positive airway pressure” or “PAP” refers to a method of respiratory ventilation used primarily in the treatment of sleep apnea. PAP ventilation is also commonly used for critically ill patients in hospital with respiratory failure, and in newborn infants (neonates). “Bi-level Positive Airway Pressure” or “BIPAP” refers to a form of temporary respiratory support for patients that have difficulty breathing. Each time the patient breathes, the BIPAP machine assists the patient by applying air pressure to the lungs while the patient is breathing out (exhaling or expiration) in order to hold open the air sacs in the lungs. “Continuous Positive Airway Pressure” or “CPAP” refers to the application of positive pressure to the airways of the spontaneously or mechanically breathing patient throughout the respiratory cycle. A CPAP machine uses continuous air pressure to produce added oxygen or simply to help keep the airways in the lungs open. The air pressure keeps the airways functioning properly and helps the individual breathe additional oxygen more easily. CPAP machines were initially used mainly by patients for the treatment of sleep apnea at home, but now are in widespread use across intensive care units as a form of ventilation.

As used herein, the term “mechanical ventilation” refers to a method to mechanically assist or replace spontaneous breathing.

As used herein, the term “external mechanical ventilation device” refers to a machine to mechanically assist or replace spontaneous breathing. Examples of external mechanical ventilation devices include, but are not limited to, hand-controlled ventilators and mechanical ventilators such as transport ventilators, ICU ventilators, and PAP ventilators (BiPAP machine, CPAP machine).

As used herein, the term “medical gas” includes gases such as compressed air, oxygen, carbon dioxide, helium, nitrogen and nitrous oxide.

As used herein, the term “one-lung ventilation”, “OLV”, “independent lung ventilation” or “ILV” consists of mechanical ventilation of a selected lung and exposure or intentional airway blocking to the other. OLV is required for a number of thoracic procedures, including, but not limited to, lung surgery, esophageal surgery, aortic surgery, mediastinal surgery, minimally invasive lung surgery, minimally invasive heart surgery, robotic heart surgery and robotic lung surgery. In a conventional OLV procedure, a double-lumen endotracheal tube, an endobronchial blocker, or a single lumen tube may be used. Double-lumen endotracheal tubes and endobronchial blockers function differently. Double-lumen endotracheal tubes isolate ventilation, separating the right and left pulmonary units using two separate endotracheal tubes. An endobronchial blocker blocks ventilation to a pulmonary segment. Endobronchial blockers are typically balloon tipped catheters that are placed in the portion of the pulmonary tree that is to be blocked (usually the right or left main stem bronchus). Ventilation to the pulmonary unit is blocked when the balloon is inflated.

As used herein, the term “positive pressure ventilation” or “PPV” refers to the process of forcing air into the lungs of a patient.

As used herein, the term “pulmonary airway” refers to those parts of the respiratory system through which air flows, conceptually beginning (on inhalation from the external environment) at the nose and mouth, and terminating in the alveoli. From the mouth or nose, inhaled air passes through the pharynx into the trachea, where the air separates into the left and right main bronchi at the carina, situated at the level of the second thoracic vertebra. The main bronchi then branch into large bronchioles, one for each lobe of the lung. Within the lobes, the bronchioles further subdivide some 20 times, ending in clusters of alveoli.

As used herein, the term “tracheal intubation” refers to the placement of a flexible plastic tube into the trachea to protect the patient's airway and provide a means of mechanical ventilation. The most common tracheal intubation is orotracheal intubation where, with the assistance of a laryngoscope, an endotracheal tube is passed through the mouth, larynx, and vocal cords, into the trachea. Another possibility is nasotracheal intubation where a tube is passed through the nose, larynx, vocal cords, and trachea.

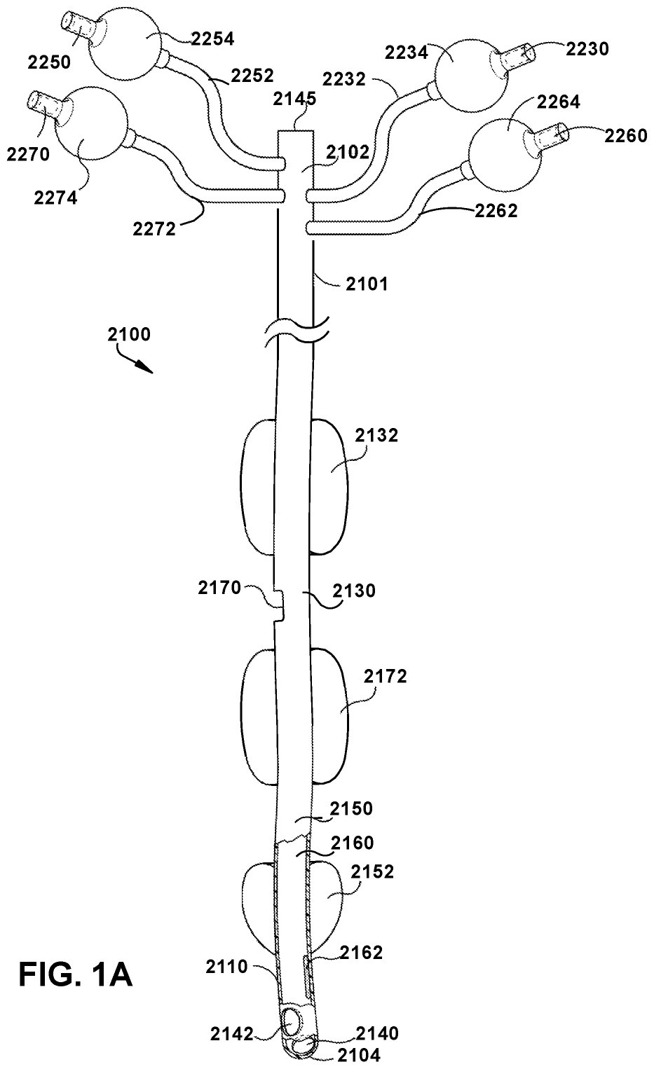

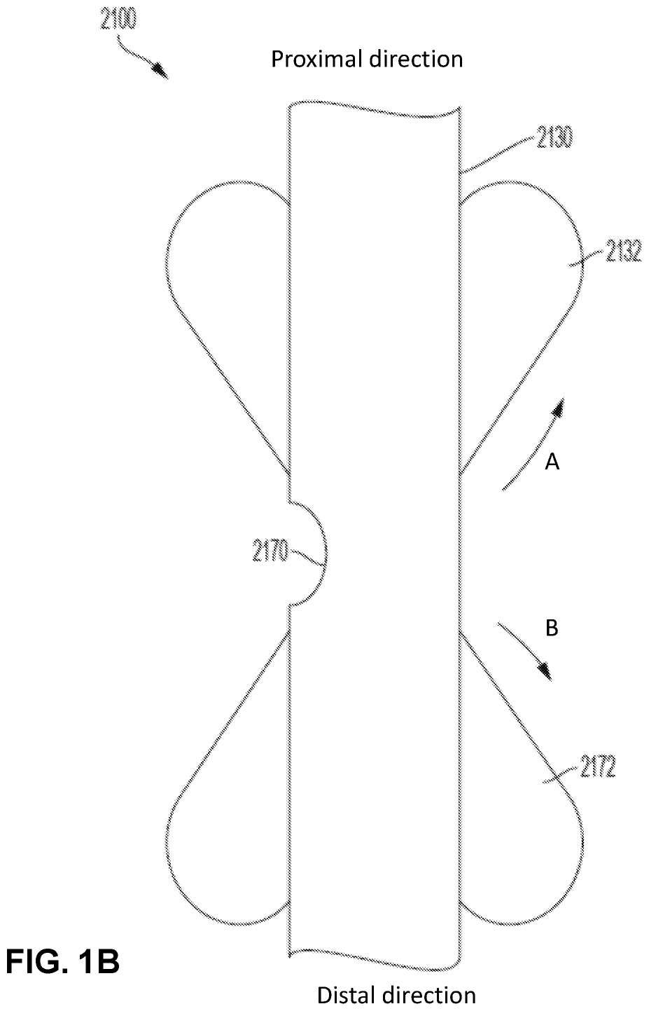

Disclosed herein are medical tubes for selective mechanical ventilation of the left lung or the right lung 1400. FIGS. 1A and 1B show example embodiments of a single lumen endobronchial tube 2100 of the present disclosure. FIG. 1A shows an embodiment of a single lumen endobronchial tube 2100 of the present disclosure. The single lumen endobronchial tube 2100 is a medical tube having a proximal end 2102, a distal end 2104, and a primary flow passage or lumen 2160 passing therebetween. The primary flow passage or lumen 2160 is a single lumen that defines a single passageway that fluidly connects an aperture 2170, a bronchial opening 2140, and, optionally, a second opening (e.g., Murphy eye 2142) with one another to selectively ventilate the left lung 1300, the right lung 1400, or both lungs of the patient. The distal end 2104 of the tube 2100 has a bronchial opening 2140. In an embodiment, the bronchial opening 2140 is smooth and beveled, thus minimizing risk of tracheal intubation airway trauma. As would be appreciated by one skilled in the art, the bronchial opening 2140 can include any shape known in the art. The single lumen endobronchial tube is specifically designed to enable an anesthesiologist to ventilate either lung (left or right), while always intubating the left lung. The distal end 2104 of the tube 2100 can optionally include a Murphy eye 2142, which is a distal opening in a wall 2110 and through an outer surface 2101 of the tube 2100 which can allow airflow in the event of the bronchial opening 2140 lying against the tracheal wall or being obstructed in other ways. Located at the proximal end 2102 of the tube 2100 is an opening 2145 sufficiently designed to connect with a mechanical ventilation device, including, but not limited to, an anesthesia machine or a PAP machine, with or without the use of an adaptor.

The tube 2100 can be manufactured to various sizes and adapted to provide mechanical ventilation to an air-breathing animal in need thereof. In an example embodiment, the tube 2100 is manufactured for human use and ranges in size from about 1.5 mm to about 11 mm in internal diameter (ID). In an embodiment, the tube 2100 is manufactured for human use and ranges in size from about 3 mm to about 10 mm in internal diameter (ID). In an embodiment, the tube 2100 is manufactured for non-human use and ranges in size from about 1.5 mm to about 40 mm in internal diameter (ID). In an embodiment, the tube 2100 is manufactured for non-human use and ranges in size from about 6 mm to about 40 mm in internal diameter (ID).

The tube 2100 may be made from a flexible material including, but not limited to, latex, silicone, polyvinyl chloride (PVC), polyurethane (PU), polytetrafluoroethylene or a similar material that has met the American National Standard for Anesthetic Equipment; ANSI Z-79 standard and implant-tested to ensure nontoxicity. In an embodiment, the tube 2100 is made from a non-toxic, clear, PVC material. In an embodiment, the tracheal portion 2130 is adapted to follow the natural contour of a patient's trachea, and the bronchial portion 2150 is adapted to follow the natural contour of a patient's left main stem bronchi. In an embodiment, to facilitate passage of the bronchial portion 2150 into the left main stem bronchi, the tube 2100 is curved or bent and resembles the shape of a hockey stick. In an embodiment, the angle of the bend is about 45°. The lumen 2160 of the tube 2100 is sized and dimensioned to allow other instrumentation to pass through the lumen 2160 as required. The removal of mucous, the injection of medication, or the insertion of fiberoptic scopes for viewing within the tube 2100 are examples of the additional instrumentation capability which is afforded by the tube 2100. In an embodiment, the single lumen endobronchial tube 2100 may be referred to as a left-sided single lumen endobronchial tube.

Continuing with FIG. 1A, in some embodiments, the tube 2100 includes a tracheal portion 2130 and a bronchial portion 2150 distal of the tracheal portion 2130. In operation, the tracheal portion 2130 is configured for placement in a trachea of a patient and the bronchial portion 2150 is configured for placement within a left main stem bronchi, providing selective ventilation of the left lung 1300 or the right lung 1300 without a need to move or reposition the tube 2100. Additionally, the tracheal portion 2130 and the bronchial portion 2150 can have a common single lumen and a common tube wall 2110 thickness. In some embodiments, a proximal end of the tracheal portion 2130 can include an opening adapted for connection to an external mechanical ventilation device, and a distal end of the bronchial portion 2150 can include an opening adapted for delivery of a medical gas to a second lung of a patient.

In some embodiments, the tube 2100 can include a first tracheal cuff 2132 located longitudinally along an exterior surface of the tracheal portion 2130 and a second tracheal cuff 2172 located longitudinally along an exterior surface of the tracheal portion 2150. The bronchial cuff 2152 can be located longitudinally along an exterior surface between the first tracheal cuff 2132 and the second tracheal cuff 2172. In an embodiment, the first tracheal cuff 2132, the second tracheal cuff 2172 and the bronchial cuff 2152 are thin walled, high volume low pressure (HVLP) balloon-like members sealed from fluid communication with the tube 2100 and adapted not to compromise the blood flow in the tracheal or bronchial wall when inflated. The first tracheal inflatable cuff 2132 can be positioned around an external surface of the tracheal portion 2130 and can be adapted to expand radially outward for sealing against a trachea of the patient. The second tracheal inflatable cuff 2172 can be positioned around an external surface of the tracheal portion 2130 and located at a position distal to the first tracheal inflatable cuff 2132, the second tracheal inflatable cuff 2172 can be adapted to expand radially outward for sealing against the trachea of the patient. The bronchial inflatable cuff 2152 can be positioned around an external surface of the bronchial portion 2150 and can be adapted to expand radially outward against the left main stem bronchi of the patient. The first tracheal cuff 2132, the second tracheal cuff 2172, and the bronchial cuff 2152 are shown in an expanded state in FIG. 1A, however, as would be appreciated by one skilled in the art, the first tracheal cuff 2132, the second tracheal cuff 2172, and the bronchial cuff 2152 can also be configured in a deflated state (not depicted).

In an embodiment, the walls of the first tracheal cuff 2132, the second tracheal cuff 2172, and the bronchial cuff 2152 are on the order of about 5 μm to about 500 μm, about 5 μm to about 250 μm, about 5 μm to about 100 μm, about 5 μm to about 50 μm, about 5 μm and about 20 μm, about 5 μm and about 15 μm. It is also contemplated that the walls may have a thickness of less than about 5 μm. Additionally, although the thickness of the walls may vary, it is desirable that the thickness of the material remain consistent throughout the cuff.

In an embodiment, the first tracheal cuff 2132, the second tracheal cuff 2172 and the bronchial cuff 2152 are tapered, spherical or elliptical in shape, although any combination of shapes is possible and within the scope and spirit of the present disclosure. For example, one, two or all three of the first tracheal cuff 2132, the second tracheal cuff 2172, and the bronchial cuff 2152 can have a tapered shape. In an example embodiment, the first tracheal cuff 2132 and the second tracheal cuff 2172 are both tapered in opposite directions with their respective tapered ends tapering toward one another (as shown with arrows A and B in FIG. 1B). As would be appreciated by one skilled in the art, in some embodiments, the first tracheal cuff 2132 and the second tracheal cuff 5152 can also be tapered in opposite directions with their respective tapered ends tapering away from one another or tapered in a same direction.

Referring to FIG. 1B, both the first tracheal cuff 2132 and the second tracheal cuff 2172 are tapered toward an aperture 2170. In this example embodiment, the opposing tapered shapes enable the endobronchial tube 2100 to better seal against the tracheal mucosa. In some embodiments, this arrangement makes the seals on the trachea more efficient without needing much inflation pressure. As the gas flows out the aperture, it builds up in the space that is formed between the trachea and the tube. With the tapered shapes, the gas flow pushes along the tapering to create a damming effect on the cuffs 2132, 2172 against the trachea providing a better seal. Additionally, the tapered cuffs 2132, 2172 can be placed sufficiently distanced from the aperture 2170 to allow passage of a suction catheter or fiberoptic scope through the aperture 2170.

Referring back to FIG. 1A, the tube 2100 can include a distal intraluminal balloon blocker 2162 adapted to inflate radially outward and deflate within the walls 2110 of the tube 2100. In particular, the intraluminal balloon blocker 2162 can be positioned along an inner surface of the tube 2100 and when inflated acts to block flow by blocking ventilation to the left main stem bronchus, for example, within the common single lumen at the distal end of the bronchial portion 2150. In an embodiment, the distal intraluminal balloon blocker 2162 is a low volume high pressure member (when the tube 2100 is placed within the patient). Various materials may be used to form the first tracheal cuff 2132, the second tracheal cuff 2172, the bronchial cuff 2152 and the distal intraluminal balloon blocker 2162. These materials include, but are not limited to, polyurethane (PU), low-density polyethylene (LDPE), polyvinyl chloride (PVC), silicone, neoprene, polyisoprene, polyamid (PA) or polyethylene teraphthalate (PETP). Additionally, copolymer admixtures for modifying the characteristics of the material may be used, for example a low-density polyethylene and ethylene-vinylacetate copolymer (LDPE-EVA), or blends of the above-mentioned materials (e.g. PU with PVC or PU with PA) would be considered suitable for forming the first tracheal cuff 2132, the second tracheal cuff 2172, the bronchial cuff 2152 and the distal intraluminal balloon blocker 2162.

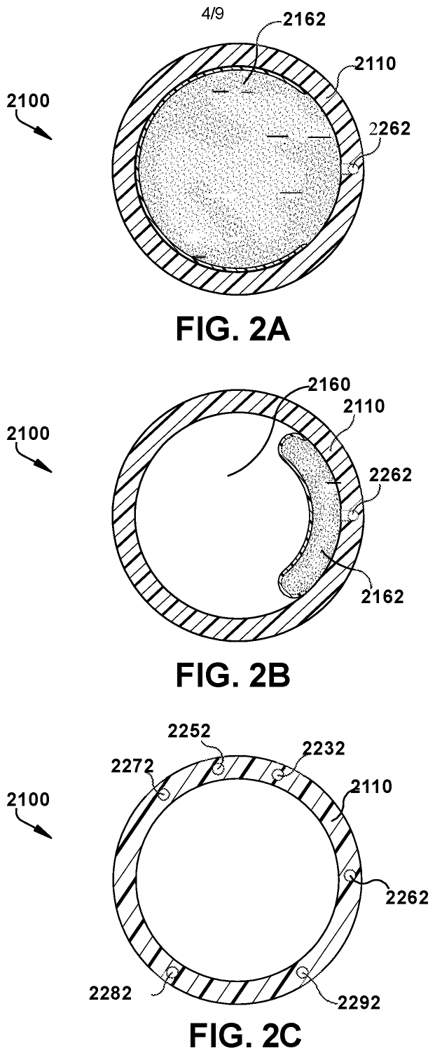

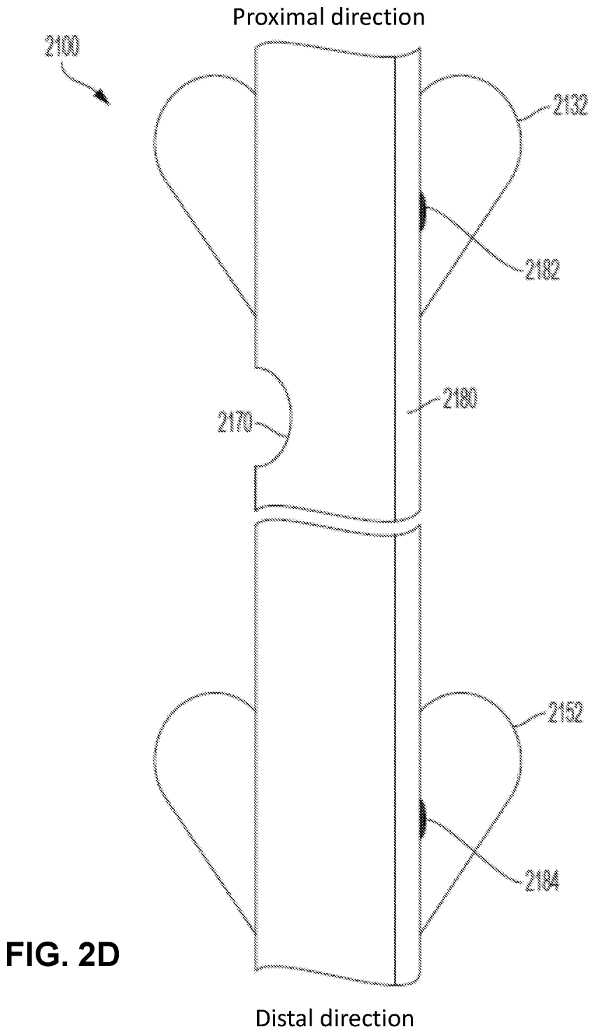

In some embodiments, the first tracheal cuff 2132, the second tracheal cuff 2172, the bronchial cuff 2152, and the distal intraluminal balloon blocker 2162 can each be remotely and selectively inflatable through a plurality of pilot tubes. In particular, the wall 2110 can have an internal wall surface, an external wall surface and a thickness therebetween configured to receive pilot tubes to inflate and deflect the first tracheal cuff 2132, the second tracheal cuff 2172, the bronchial cuff 2152, and the distal intraluminal balloon blocker 2162. FIGS. 2A-2C show a plurality of pilot tubes 2232, 2272, 2252 and 2262, respectively, running longitudinally through the wall 2110 of the tube 2100. Referring to FIG. 1A, each pilot tube 2232, 2272, 2252 and 2262 emerges from the outer surface 2101 of the tube 2100 near the proximal end 2102 of the tube 2100. Attached to a proximal end of each pilot tube 2232, 2272, 2252 and 2262 is a non-return valve 2230, 2270, 2250 and 2260 which is adapted to receive the nozzle of a syringe (not visible) and a complementary indicator bladder 2234, 2274, 2254 and 2264 which enables an anesthesiologist to confirm that each of the first tracheal cuff 2132, the second tracheal cuff 2172, the bronchial cuff 2152, and the distal intraluminal balloon blocker 2162 has been inflated or deflated. The non-return valves 2230, 2270, 2250 and 2260 may be attached to a syringe for injecting a predetermined quantity of gas or fluid.

Continuing with FIG. 2A, FIG. 2B and FIG. 2C, the figures show bisected views of the tube 2100 with different example configurations of the pilot tubes 2232, 2272, 2252 and 2262 within the wall 2110. FIG. 2A shows the tube 2100 with the intraluminal balloon blocker 2162 in an inflated state and the pilot tube 2262 that controls the inflation/deflation of said intraluminal balloon blocker 2162. FIG. 2B shows the tube 2100 with the intraluminal balloon blocker 2162 in a deflated state and the pilot tube 2262 that controls the inflation/deflation of said intraluminal balloon blocker 2162, exposing the lumen 2160. FIG. 2C shows the tube 2100 with the pilot tubes 2232, 2272, 2252 and 2262 within the wall 2110 of the tube 2100 configured to control inflation and deflation of the first tracheal cuff 2132, the second tracheal cuff 2172, the bronchial cuff 2152, and the distal intraluminal balloon blocker 2162, respectively. In some embodiments, the independent pilot tubes 2232, 2272, 2252 and 2262 for each cuff/blocker can be used to control inflation pressures, so the pressures can be more specifically measured and monitored for each cuff/blocker. Additionally, FIG. 2C shows conduits 2282 and 2292 running longitudinally through the wall 2110 of the tube 2100 is configured to deliver gas to a patient at positive pressure in order to hold open alveoli that would normally close at the end of expiration (e.g., for use with a PAP machine).

In some embodiments, the endobronchial tube 2100 can include a single inflation channel 2180 for inflating and deflating a combination of the first tracheal cuff 2132 and the bronchial cuff 2152. FIG. 2D depicts a single channel configured to control inflation of the first tracheal cuff 2132 and the bronchial cuff 2152. The single channel provides a simplified design for manufacture by having one channel inflate the first tracheal cuff 2132 and the bronchial cuff 2152, which in some embodiments, are always in the inflated position. In particular, FIG. 2D shows a side view of the tube 2100 with the single inflation channel 2180 within the wall 2110. Additionally, the inflation channel 2180 can be configured with fill ports 2182, 2184 that are in open communication with the first tracheal cuff 2132 and the bronchial cuff 2152, located adjacent to the fill ports 2182, 2184. In operation, the fill ports 2182, 2184 are configured to allow air or fluid to pass to and from the inflation channel 2180 into the respective first tracheal cuff 2132 and bronchial cuff 2152 to inflate/deflate the first tracheal cuff 2132 and the bronchial cuff 2152 substantially simultaneously, as desired. The single inflation channel allows both the first tracheal cuff 2132 and the bronchial cuff 2152 to be filled at the same time, which may be desirable for particular operations (e.g., when limiting flow from the endobronchial tube 2100 to the right lung 1400 via the aperture 2170).

In some embodiments, an aperture 2170 is provided through the wall 2110 of the tube 2100 between the first tracheal balloon cuff 2132 and the second tracheal balloon cuff 2172. The aperture 2170 can be adapted to deliver medical gas to a second lung of the patient and the aperture 2170 is positioned sufficiently close to the second tracheal inflatable cuff 2172 such that when the second tracheal inflatable cuff 2172 is inflated, the second tracheal inflatable cuff 2172 blocks the aperture 2170 to control amount of the medical gas passing through the aperture 2170. The aperture 2170 can be of any shape or size. In some embodiments, additional fenestrations can be added near the aperture 2170, as needed, to ensure equal flow to both lungs in circumstances in which both lungs are being ventilated. In this way, structural integrity can be assured in case the aperture 2170 has to be made too large and possibly compromise the structure of the tube 2100. For example, if the aperture 2170 has been created with increased surface area for desired flow dynamics. In an example embodiment, the aperture 2170 can be beveled to allow passage of a suction catheter or fiberoptic scope through the aperture 2170.

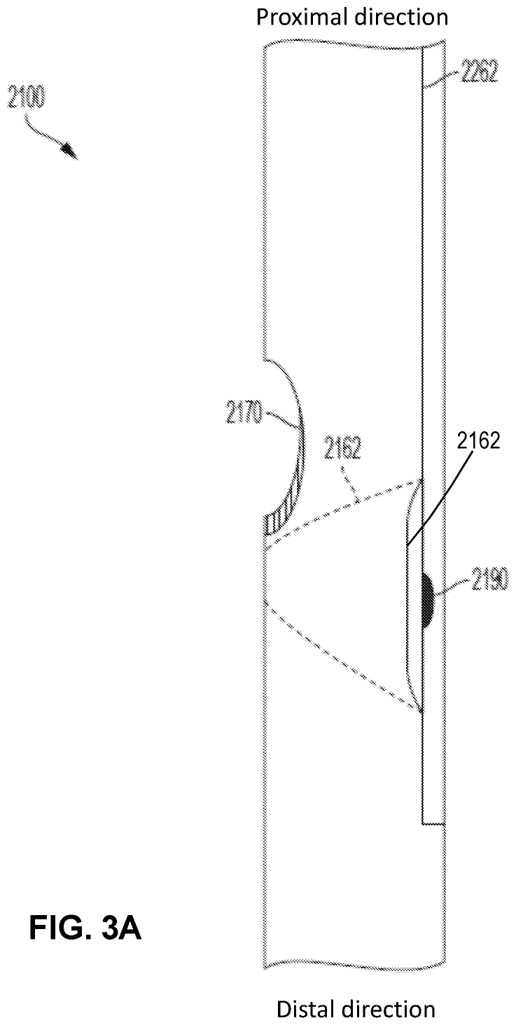

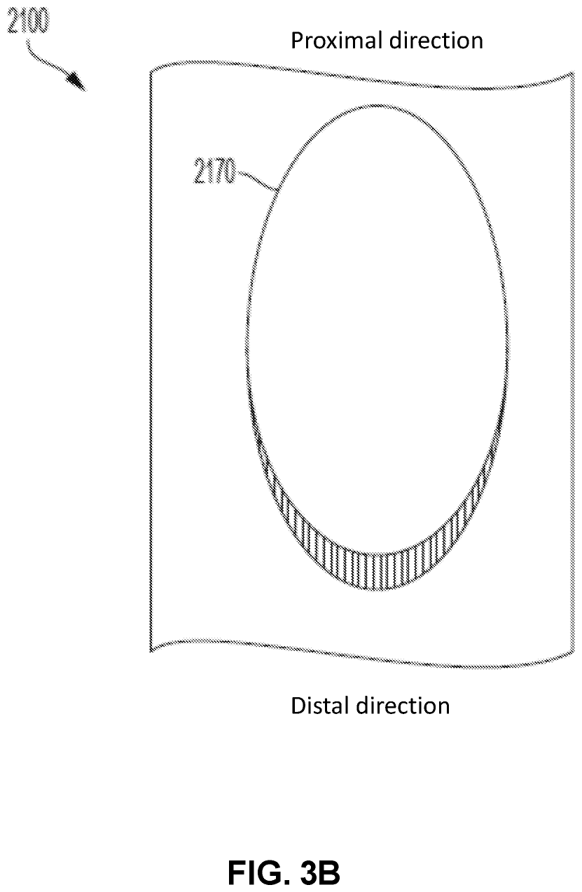

FIG. 3A and FIG. 3B show an example embodiment of a beveled aperture 2170, where the bevel is created in the wall of the endobronchial tube 2100. The beveling can be done in a semi lunar fashion where the interior aspect of the tube wall is at about a 45-degree angle (35-degree to 55-degree) to the exterior aspect of the tube wall. In this manner, an apparatus that is passed through the aperture is less likely to get caught on the distal lip of the aperture. The actual edges can be as rounded and smooth as possible. In particular, FIG. 3B shows a front view of the beveled aperture 2170. The beveled aperture can be beveled along the end of the aperture 2170 nearest to the distal end of the tube 2100. As would be appreciated by one skilled in the art, the aperture 2170 can be beveled along the entire circumference of the aperture 2170 or only a portion thereof (e.g., as depicted in FIG. 3B).

Additionally, the aperture 2170 can be dimensioned so that a fiberoptic scope can pass through the aperture 2170. As shown in FIG. 3A, the intraluminal balloon blocker 2162 may be placed immediately distal to the aperture 2170 with sufficient space to allow passage of a suction catheter or fiberoptic scope through the aperture 2170, while the balloon blocker 2162 is inflated or deflated. In particular, the aperture 2170 can be located just proximal to the balloon blocker 2162, so that an inflated balloon blocker 2162 can help guide instrumentation (such as a suction catheter or fiberoptic scope) from the lumen 2160 out the aperture 2170. Moreover, when the second endotracheal cuff is deflated, it can allow for suction or inspection of the right lung 1400. In an embodiment, the balloon blocker 2162 is spherical or elliptical in shape, although any desired shape is possible and within the scope and spirit of the present disclosure. For example, the balloon blocker 2162 can be semicircular with the dome at or just below the aperture 2170.

The present disclosure provides a method of selective ventilation of a patient. The method can include inserting a single lumen endobronchial tube into the pulmonary airway of a patient. The tube can be placed so the tracheal portion of the tube is in a trachea of the patient and the bronchial portion of the tube is within a left main stem bronchi of the patient. As discussed above, in some embodiments, the tube can include a lumen extending throughout the tube's entire length with an opening at each of opposed distal and proximal ends of the tube, the opening at the proximal end of the tube being adapted for connection to an external mechanical ventilation device, and the opening at the distal end of the tube being adapted for delivery of a medical gas; a distal bronchial cuff positioned along the external wall surface and adapted to expand radially outward; a proximal tracheal cuff positioned along the external wall surface and adapted to expand radially outward; and a distal intraluminal balloon blocker at a distal location of the tube, distal to the aperture; positioning the tube in the pulmonary airway such that the distal bronchial cuff is in the left main stem bronchus, and the proximal tracheal cuff is in the trachea. In some embodiments, the tube can include one or more markers conveying to a user where to stop the insertion of the tube. The left lung or the right lunch can be selectively ventilated without moving or repositioning the tube. To selectively ventilate the left lung of the patient, inflate the first tracheal inflatable cuff, the second tracheal inflatable cuff, and the bronchial inflatable cuff and deflate the intraluminal balloon blocker. To selectively ventilate the right lung of the patient, inflate the first tracheal inflatable cuff, the bronchial inflatable cuff, and the intraluminal balloon blocker and deflate the second tracheal inflatable cuff. Of course, both lungs can be ventilated at the same time as needed by, for example, inflating the first second tracheal cuff and, possibly, the branchial inflatable cuff.

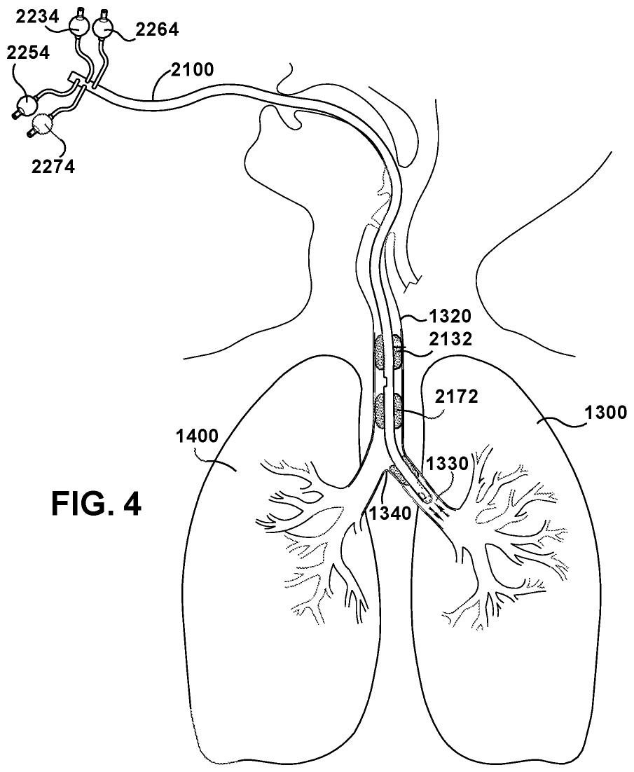

In operation, as shown in FIG. 4, the tube 2100 is utilized for selective mechanical ventilation of a left or right lung. In particular, upon placement of the tracheal portion 2130 of the tube 2100 in a trachea of the patient and the bronchial portion 2150 within a left main stem bronchi, selective ventilation of the left lung 1300 or the right lung 1400 is achievable without a need to move or reposition the tube 2100, wherein to selectively ventilate the left lung 1300 of the patient, the first tracheal inflatable cuff, 2132 the second tracheal inflatable cuff 2172, and the bronchial inflatable cuff 2152 are placed in an inflated position and the intraluminal balloon blocker 2162 is placed in a deflated position, and to selectively ventilate the right lung 1400 of the patient, the first tracheal inflatable cuff, 2132, the bronchial inflatable cuff 2152, and the intraluminal balloon blocker 2162 are placed in the inflated position and the second tracheal inflatable cuff 2172 is placed in the deflated position.

Referring to FIG. 4, the single lumen endobronchial tube 2100 is positioned within a patient to facilitate artificial ventilation of the respiratory system. The single lumen endobronchial tube 2100 has been placed within a mouth of the patient and positioned such that the tracheal portion 2130 resides within the trachea 1320 and the bronchial portion 2150 resides within the left main stem bronchi 1330. The tube 2100 may be sufficiently designed such that the bronchial portion 2150 curves for ease of placement beyond the carina 1340 into the left main stem bronchi 1330. In this placement, ventilation of the left lung or the right lung can be accomplished without having to move the lumen endobronchial tube. Placement of the lumen endobronchial tube can be performed with or without fiberoptic visualization. Although FIG. 4 shows the lumen endobronchial tube being inserted through the mouth of the patient, it should be understood that the lumen endobronchial tube can also be inserted through the nasal passages into the airway passage.

Once proper positioning of the lumen endobronchial tube in the pulmonary airway is determined, the bronchial cuff 2152 is inflated to a desired pressure by pushing a fluid such as air or saline through the pilot tube 2252. In an embodiment, the bronchial cuff 2152 is inflated so that the bronchial cuff pressure (BCP) is in the range of about 15 cm H₂O (about 11 mm Hg) to about 30 cm H₂O (about 22 mm Hg). The first tracheal cuff 2132 is inflated by pushing a fluid such as air or saline through the pilot tube 2232 or inflation channel 2180 leading to the first tracheal cuff 2132. In some embodiments, as discussed above, the single inflation channel 2180 can be employed to inflate both first tracheal cuff 2132 and the bronchial cuff 2152 simultaneously. The second tracheal cuff 2172 is inflated by pushing a fluid such as air or saline through the pilot tube 2272 leading to the second tracheal cuff 2172. In an embodiment, the first tracheal cuff 2132 and the second tracheal cuff 2172 are inflated so that the cuff pressure is in the range of about 15 cm H₂O (about 11 mm Hg) to about 30 cm H₂O (about 22 mm Hg). The seal formed by the inflated tracheal cuffs 2132 and 2172 are adapted to substantially provide a seal between the outside of the lumen endobronchial tube and the interior of the trachea 1320 in which the tube 2100 is inserted.

Thereafter, the desired agent(s) are then introduced, for example from an anesthesia machine, through the lumen 2160 of the tube 2100 to deliver the desired agent(s) to the left lung 1300. The closed space between the first tracheal cuff 2132 and the second tracheal cuff 2172 is adapted to block entry of the desired agent(s) to the right lung 1400. If the desired agent(s) are to be delivered into the right lung 1400 and not the left lung 1300, the procedure can proceed as follows: the second tracheal cuff 2172 is deflated, and the distal intraluminal balloon blocker 2162 is inflated by pushing a fluid such as air or saline through the pilot tube 2262 leading to the distal intraluminal balloon blocker 2162. In an embodiment, the distal intraluminal balloon blocker 2162 is inflated so that the cuff pressure is in the range of about 20 cm H₂O (about 14.7 mm Hg) to about 95 cm H₂O (about 69 mm Hg). The inflated distal intraluminal balloon blocker seals the lumen 2160 of the tube 2100 distal to the inflated distal intraluminal balloon blocker 2162 such that sufficient blockage of the agents to the left lung 1300 is achieved.

It is also contemplated that in an alternative embodiment of the single lumen endobronchial tube, the distal intraluminal balloon blocker 2162 (as well as the other co-codependent components of the distal intraluminal balloon blocker 2162 including the pilot tube 2262, the non-return valve 2260 and the pilot balloon 2264) are absent. In such an embodiment, a conventional endobronchial blocker can be used to block ventilation of the left main stem bronchi.

Endobronchial tube displacement may result in life-threatening complications and continuous direct vision of the position of the endobronchial tube may enable safer management. In an embodiment, any of the single lumen endobronchial tubes disclosed herein may further include a built-in video camera having an optional built-in light source. The video camera is connected to a monitor via a cable that runs longitudinally through the wall of the tube. The video camera and cable are embedded within the common tube wall. In an embodiment, the view from the video camera appears continuously on the monitor in the anaesthetist's vicinity. In an embodiment, the video camera terminates at a location that is distal to the aperture that is provided through the wall of the tube between the tracheal balloon cuff and the bronchial balloon cuff. The placement of the video camera at this location may provide for a better view of the carina of the trachea, the cartilaginous ridge within the trachea that runs anteroposteriorly between the two primary bronchi at the site of the tracheal bifurcation at the lower end of the trachea. This may help ensure that the bronchial portion of the single lumen endobronchial tube is positioned below the carina. In embodiments where the single lumen endobronchial tube includes a built-in video camera, it may not be necessary to use a fiberoptic scope during placement, use, or removal of the tube, discussed in greater detail with respect to U.S. application Ser. No. 14/082,664, incorporated herein by reference in its entirety.

In an embodiment, a single lumen endobronchial tube of the present disclosure can be used in general anesthesia, intensive care, and emergency medicine for airway management and mechanical ventilation. In an embodiment, a single lumen endobronchial tube of the present disclosure can be used during any procedure where lung separation is necessary to isolate and selectively ventilate a single lung, including, but not limited to, thoracic surgical procedures, lung abscess surgical procedures, and pulmonary hemorrhage surgical procedures. In some embodiments, a single lumen endobronchial tube of the present disclosure is used with a BiPAP machine. In some embodiments, a single lumen endobronchial tube of the present disclosure is used with a CPAP machine. In such embodiments, the proximal end of the medical tube is connected to the PAP machine such that compressed air is delivered directly to the pulmonary airway of a patient. Use of a single lumen endobronchial tube of the present disclosure in conjunction with a CPAP machine may be useful in treating or preventing various conditions in patients, including, but not limited to, obstructive sleep apnea and respiratory failure.

In some embodiments, a single lumen endobronchial tube of the present disclosure is used with an anesthesia machine. In such embodiments, the proximal end of the medical tube is connected to the anesthesia machine such that medical gases are delivered to the pulmonary airway of an air-breathing animal. Use of a single lumen endobronchial tube of the present disclosure in conjunction with an anesthesia machine may be useful to support the administration of anesthesia to the animal.

All patents, patent applications, and published references cited herein are hereby incorporated by reference in their entirety. It will be appreciated that various of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. An endobronchial tube comprising:
a medical tube comprising an endotracheal portion and a bronchial portion having a common single lumen extending between a proximal end and a distal end of the tube, wherein the proximal end of the endotracheal portion includes a first opening adapted for connection to an external mechanical ventilation device and the distal end of the bronchial portion includes a second opening adapted for delivery of a medical gas;
a first endotracheal inflatable cuff positioned around an external surface of the endotracheal portion and adapted to expand radially outward for sealing against a trachea of a patient;
a second endotracheal inflatable cuff positioned around an external surface of the endotracheal portion and located at a position distal to the first endotracheal inflatable cuff, the second endotracheal inflatable cuff adapted to expand radially outward for sealing against the trachea of the patient,
wherein the first endotracheal inflatable cuff and the second endotracheal inflatable cuff are tapered;
a bronchial inflatable cuff positioned around an external surface of the bronchial portion located distal to the first endotracheal inflatable cuff and the second endotracheal inflatable cuff, the bronchial inflatable cuff adapted to expand radially outward against the left main stem bronchi of the patient;
an aperture positioned between the first tracheal inflatable cuff and the second tracheal inflatable cuff and adapted to deliver an amount of medical gas to the second lung of the patient; and
an intraluminal balloon blocker positioned along an inner surface of the bronchial portion distal to the aperture and adapted to expand for sealing the common single lumen at the distal end of the bronchial portion,
wherein the single lumen defines a single passageway that fluidly connects the aperture, the first opening, and the second opening with one another to selectively ventilate the left lung, the right lung, or both lungs of the patient.

2. The endobronchial tube of claim 1, wherein the first endotracheal inflatable cuff, the second endotracheal inflatable cuff, the bronchial inflatable cuff and the intraluminal balloon blocker are each remotely and selectively inflatable.

3. The endobronchial tube of claim 1, wherein the first endotracheal inflatable cuff and the second endotracheal inflatable cuff are tapered in opposite directions toward the aperture.

4. The endobronchial tube of claim 1, wherein the first endotracheal inflatable cuff and the bronchial inflatable cuff are inflatable and deflatable via a single inflation channel.

5. The endobronchial tube of claim 1, wherein the intraluminal balloon blocker is immediately distal to the aperture.

6. The endobronchial tube of claim 5, wherein the aperture is beveled.

7. The endobronchial tube of claim 1, wherein upon placement of the tube in a trachea of the patient and the left main stem bronchi, selective ventilation of the left lung or the right lung is achievable without a need to move or reposition the tube, wherein to selectively ventilate the left lung of the patient, the first tracheal inflatable cuff, the second tracheal inflatable cuff, and the bronchial inflatable cuff are placed in an inflated position and the intraluminal balloon blocker is placed in a deflated position, and to selectively ventilate the right lung of the patient, the first tracheal inflatable cuff, the bronchial inflatable cuff and the intraluminal balloon blocker are placed in the inflated position and the second tracheal inflatable cuff is placed in the deflated position

8. An endobronchial tube comprising:
a medical tube comprising an endotracheal portion and a bronchial portion having a common single lumen extending between a proximal end and a distal end of the tube, wherein the proximal end of the endotracheal portion includes a first opening adapted for connection to an external mechanical ventilation device and the distal end of the bronchial portion includes a second opening adapted for delivery of a medical gas;
a first endotracheal inflatable cuff positioned around an external surface of the endotracheal portion and adapted to expand radially outward for sealing against a trachea of a patient;
a second endotracheal inflatable cuff positioned around an external surface of the endotracheal portion and located at a position distal to the first endotracheal inflatable cuff, the second endotracheal inflatable cuff adapted to expand radially outward for sealing against the trachea of the patient;
a bronchial inflatable cuff positioned around an external surface of the bronchial portion located distal to the first endotracheal inflatable cuff and the second endotracheal inflatable cuff, the bronchial inflatable cuff adapted to expand radially outward against the left main stem bronchi of the patient;
a beveled aperture positioned between the first tracheal inflatable cuff and the second tracheal inflatable cuff and adapted to deliver an amount of medical gas to the second lung of the patient; and
a distal intraluminal balloon blocker positioned distal to the aperture and adapted to expand radially outward sealing the common single lumen,
wherein the single lumen defines a single passageway that fluidly connects the beveled aperture, the first opening, and the second opening with one another to selectively ventilate the left lung, the right lung, or both lungs of the patient.

9. The endobronchial tube of claim 8, wherein the first endotracheal inflatable cuff and the second endotracheal inflatable cuff are tapered in opposite directions toward the aperture.

10. The endobronchial tube of claim 8, wherein the distal intraluminal balloon blocker is a low volume high pressure member.

11. The endobronchial tube of claim 8 wherein the tracheal inflatable cuff, the bronchial inflatable cuff and the distal intraluminal balloon blocker are each remotely and selectively inflatable.

12. The endobronchial tube of claim 8, further comprising a second tracheal inflatable cuff positioned around an external surface of the tracheal portion and adapted to expand radially outward at a respective distal location relative to the aperture.

13. The endobronchial tube of claim 8, further comprising a built-in video camera embedded within the common tube wall.

14. The endobronchial tube of claim 8, wherein upon placement of the tube in a trachea of the patient and the left main stem bronchi, selective ventilation of the left lung or the right lung is achievable without a need to move or reposition the tube, wherein to selectively ventilate the left lung of the patient, the first tracheal inflatable cuff, the second tracheal inflatable cuff, and the bronchial inflatable cuff are placed in an inflated position and the intraluminal balloon blocker is placed in a deflated position, and to selectively ventilate the right lung of the patient, the first tracheal inflatable cuff, the bronchial inflatable cuff and the intraluminal balloon blocker are placed in the inflated position and the second tracheal inflatable cuff is placed in the deflated position

15. A method for one-lung ventilation of a lung comprising:
inserting a single lumen endobronchial tube into in a trachea of a patient and a left main stem bronchi of a left lung of the patient, the single lumen endobronchial tube comprising:
a medical tube comprising an endotracheal portion and a bronchial portion having a common single lumen extending between a proximal end and a distal end of the tube, wherein the proximal end of the endotracheal portion includes a first opening adapted for connection to an external mechanical ventilation device and the distal end of the bronchial portion includes a second opening adapted for delivery of a medical gas;
a first endotracheal inflatable cuff positioned around an external surface of the endotracheal portion and adapted to expand radially outward for sealing against a trachea of a patient;
a second endotracheal inflatable cuff positioned around an external surface of the endotracheal portion and located at a position distal to the first endotracheal inflatable cuff, the second endotracheal inflatable cuff adapted to expand radially outward for sealing against the trachea of the patient, wherein the first endotracheal inflatable cuff and the second endotracheal inflatable cuff are tapered;
a bronchial inflatable cuff positioned around an external surface of the bronchial portion distal to the first endotracheal inflatable cuff and the second endotracheal inflatable cuff, the bronchial inflatable cuff adapted to expand radially outward against the left main stem bronchi of the patient;
an aperture positioned between the first tracheal inflatable cuff and the second tracheal inflatable cuff and adapted to deliver an amount of medical gas to the second lung of the patient,
an intraluminal balloon blocker positioned along an inner surface of the bronchial portion distal to the aperture and adapted to expand for sealing the common single lumen at the distal end of the bronchial portion; and
wherein the single lumen defines a single passageway that fluidly connects the beveled aperture, the first opening, and the second opening with one another to selectively ventilate the left lung, the right lung, or both lungs of the patient;
positioning the tube in the pulmonary airway of a patient such that the di bronchial inflatable cuff is in the left main stem bronchus, and the first endotracheal cuff and the second endotracheal inflatable cuff are in the trachea;
connecting the proximal end of the medical tube to the external mechanical ventilation device;
inflating the bronchial inflatable cuff radially outwardly to seal against the surrounding bronchus of the left lung; and
inflating at least one of the first endotracheal inflatable cuff and the second endotracheal inflatable cuff radially outwardly to seal against the surrounding trachea of the patient.

16. The endobronchial tube of claim 15, wherein the first endotracheal inflatable cuff and the second endotracheal inflatable cuff are tapered in opposite directions toward the aperture.

17. The endobronchial tube of claim 16, wherein the aperture is beveled.

18. The method of claim 15, wherein the tube further comprises a built-in video camera embedded within the tube wall for real-time visualization of the method.

19. The method of claim 15, wherein upon insertion of the tube into the left main stem bronchi, selective ventilation of the left lung or the right lung is achievable without a need to move or reposition the tube, wherein to selectively ventilate the left lung of the patient, the first tracheal inflatable cuff, the second tracheal inflatable cuff, and the bronchial inflatable cuff are placed in an inflated position and the intraluminal balloon blocker is placed in a deflated position, and to selectively ventilate the right lung of the patient, the first tracheal inflatable cuff, the bronchial inflatable cuff and the intraluminal balloon blocker are placed in the inflated position and the second tracheal inflatable cuff is placed in the deflated position.

20. The method of claim 15, wherein the intraluminal balloon blocker is immediately distal to the aperture.
